(19) United States
(12) Patent Application Publication
(10) Pub. No.: US 2008/0281150 A1
(43) Pub. Date: Nov. 13, 2008

(54) Suction Cup Epicardial Stabilizer Device for Cardiac Surgery

(75) Inventor: John T. M. Wright, Denver, CO (US)

Correspondence Address: SWANSON & BRATSCHUN, L.L.C., 8210 SOUTHPARK TERRACE, LITTLETON, CO, 80120, US

(73) Assignee: Genesee BioMedical, Inc, Denver, CO (US)

(21) Appl. No.: 11/568,022

(22) PCT Filed: Apr. 20, 2005
(86) PCT No.: PCT/US05/13383

§ 371 Date: Oct. 17, 2006

Related U.S. Application Data
(60) Provisional application No. 60/563,680, filed on Apr. 20, 2004.

(51) Int. Cl. A61B 17/00

(52) U.S. Cl. 600/37

(57) ABSTRACT

An epicardial stabilizer foot comprises a frame configured to reside adjacent to a target portion of a beating heart and a number of self-retaining flexible suction cups attached to the frame for adhering the frame to the surface of the beating heart adjacent the target portion. The frame may be configured to partially surround the target portion with the associated suction cups surrounding the target portion as well. A method for stabilizing a target portion of a beating heart comprises pressing the suction cups of the suction cup stabilizer foot adjacent the target portion of a beating heart to form a partial vacuum attachment therebetween and fixing the position of the frame to prevent motion of the frame relative to the beating heart.

### TECHNICAL FIELD

The present invention is directed toward surgical tools including surgical tools for cardiac surgery, and more particularly toward an epicardial stabilizer foot for cardiac surgery.

### BACKGROUND ART

Off-Pump Coronary Artery Bypass (OPCAB) is coronary surgery that is carried out on the beating heart. Prior to the advent of OPCAB surgery the patient was placed on cardiopulmonary bypass, using a heart lung machine including a cardiotomy reservoir, oxygenator associated connecting tubing and more. Following the commencement of total cardiopulmonary bypass the heart was arrested. The surgeon was then able to operate on a still, and flaccid heart. The OPCAB technique has evolved over the past decade, and started to find increasing acceptance from around 1997 and onwards. Currently, it is estimated that approximately 20%-25% of all coronary bypass surgeries are using OPCAB techniques.

While a minority of surgeons use OPCAB exclusively, or nearly so, the majority of surgeons using OPCAB use the technique as an adjunct to conventional coronary artery bypass with cardio-pulmonary bypass support. The driving force that brought this operative change includes a reduction in post-operative complications due to embolism or micro-embolism; and the potential to shorten hospital and recovery time and hence reduce overall costs of the treatment procedure.

To more easily and accurately bypass a coronary artery on the beating heart, the immediate surface of the heart surrounding the anastomotic site must be rendered relatively akinetic. Stabilization of this local area may be achieved by placing a stabilizing foot, attached to a suitably rigid fixture (usually to a sternal retractor), on the surface of the heart partially surrounding the anastomotic site. An arm (articulated or otherwise) is firmly attached between the stabilization foot and the sternal retractor, thus fixing and immobilizing the stabilization foot relative to the general position of the heart. A more detailed description of a representative support arm and a procedure for stabilization can be found in Nguyen, U.S. Patent Application Publication No. US 2003-0158542 A1, the contents of which are expressly incorporated by reference herein.

Currently available stabilization feet include:

a) Rigid “U” shaped mechanical foot (Guidant ACROBAT™ mechanical off-pump system, Genesee BioMedical ANASTOSURE™ stabilizer, and similar mechanical friction feet from Estech, US Surgical and Genzyme). A foot having a high friction face is pushed onto the surface of the heart, and the friction forces generated between the foot and the surface of the heart renders the area of the heart within the area of the foot relatively akinetic.

b) Active vacuum, multi-port foot connected via a flexible tube to a controlled vacuum source (Medtronic Octopus® 4.3 Tissue Stabilizer, Guidant ACROBAT™ SUV Vacuum Off-Pump System). The foot is place onto the heart, the vacuum is turned on and the partial vacuum in the space between the surface of the heart and the vacuum port of the epicardial stabilizer causes the stabilizer to cling to the surface of the heart, generating the stabilization force. These devices have the disadvantage that they require an external vacuum source and vacuum regulator, vacuum tubing and blood trap, all which add to the complexity and cost of the procedure.

The majority of the devices currently marketed for myocardial stabilization are single patient use. Usually, the arm and the foot are disposable. One exception is the Genesee BioMedical LocNess® Tissue stabilizer system that utilizes a reusable arm and disposable Anastosure™ mechanical epicardial stabilizer feet, described in U.S. Publication No. US 2003-0158542 A1.

The stabilizer feet currently available rely solely upon friction forces or require connection to a vacuum source to secure the epicardial surface of a beating heart. Those dependent on friction require considerable downward force on the heart that can be difficult to maintain. Those requiring a vacuum source implicate tubes and equipment that can crowd an operating theater.

The present invention is intended to overcome one or more of the problems associated with the stabilizer feet discussed above.

### SUMMARY OF THE INVENTION

A first aspect of the invention is an suction stabilizer foot comprising a frame configured to reside adjacent to a target portion of a beating heart and a number of self retaining flexible suction cups attached to the frame for adhering the frame to the surface of the beating heart adjacent the target portion. In one embodiment, the frame partially surrounds the target portion and the associated suction cups partially surround the target portion as well. In such an embodiment the frame may comprise two arms extending substantially in parallel joined at their proximal ends to a bridge extending therebetween. The suction cups are spaced lengthwise of each of the extending arms. The arms may be canted relative to each other along a lengthwise axis to define a substantially concave configuration therebetween or the arms may be coplanar. The frame may be malleable to allow a surgeon to conform the frame to the surface of the heart adjacent a target portion.

A second aspect of the invention is a method for stabilizing a target portion of a beating heart. A frame having at least two suction cups attached thereto is provided. The suction cups are pressed onto tissue adjacent the target portion of the beating heart to form a partial vacuum attachment therebetween. The position of the frame is fixed to prevent motion of the frame. The method may further include providing suture stays spaced along the frame and pressing the suction cups to partially surround a lengthwise portion of the target artery. Sutures are inserted around axially spaced lengthwise portions of the target artery to form a loop around each axially spaced lengthwise portion of the target artery. The sutures are tensioned to occlude the lengthwise portions of the target artery and attached to the suture stays. The frame may comprise two elongate arms extending substantially in parallel joined at proximal ends to a bridge extending therebetween, at least two of the plurality of suture stays and at least two suction cups being spaced along the length of each lengthwise arm.

### BRIEF DESCRIPTION OF THE DRAWINGS

FIG. 1 is a perspective view of a sternal retractor having a support arm clamped thereto with a suction cup stabilizer foot in accordance with the present invention attached to a distal end of the support arm;

FIG. 2 is a perspective view of the suction cup stabilizer foot of FIG. 1;

FIG. 3 is a plan view of the suction cup stabilizer foot of FIG. 2;

FIG. 4 is a cross-section of the suction cup stabilizer foot taken along line A-A of FIG. 3;

FIG. 5 is a perspective view of a second embodiment of a suction cup stabilizer foot in accordance with the present invention;

FIG. 6 is a plan view of the second embodiment of the suction cup stabilizer foot of FIG. 5;

FIG. 7 is a cross-section of the second embodiment of the suction cup stabilizer foot taken along line B-B of FIG. 6;

FIG. 8 is a cross-section of a suction cup stabilizer leg taken along line A-A of FIG. 3 showing an alternate embodiment of an attachment button allowing for gimbaled movement of the suction cup relative to a frame;

FIG. 9. is a perspective view of a third embodiment of a suction cup stabilizer foot;

FIG. 10 is a fourth embodiment of a suction cup stabilizer foot;

FIG. 11 is a fifth embodiment of a suction cup stabilizer foot;

FIG. 12 is a cross-section of the suction cup stabilizer as shown in FIG. 4 attached to an epicardial surface of a heart.

### DETAILED DESCRIPTION OF THE PREFERRED EMBODIMENT

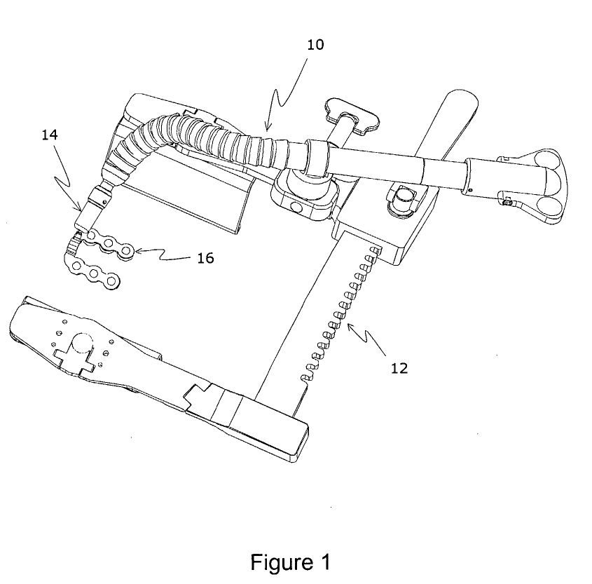

FIG. 1 is a perspective view of a support arm 10 for assisting in the performance of cardiac surgery attached to a blade arm of a sternal retractor 12. Attached at a distal end 14 of the support arm 10 is a suction cup stabilizer foot 16.

The support arm 10 is described in detail in U.S. Patent Application Publication No. US 2003-0158542 A1. The support arm 10 is intended for connection to a sternal retractor 12 as illustrated in FIG. 1 and the support arm 10 facilitates stationary or fixed placement of suction cup stabilizer foot 16 relative to a patient's heart to immobilize or stabilize a target portion of a beating heart through a procedure described in Publication No. US 2003-0158542 A1. While this application speaks to the suction cup stabilizer foot for use in association with the stabilization of a target portion of a beating heart, it should be appreciated by those of skill in the art that the suction cup stabilizer foot could be used for stabilization of other tissue in a human or other animal in conjunction with a support arm and sternal retractor or any other support fixture that allows the suction cup stabilizer foot 16 to be stationary or fixed relative to the target tissue.

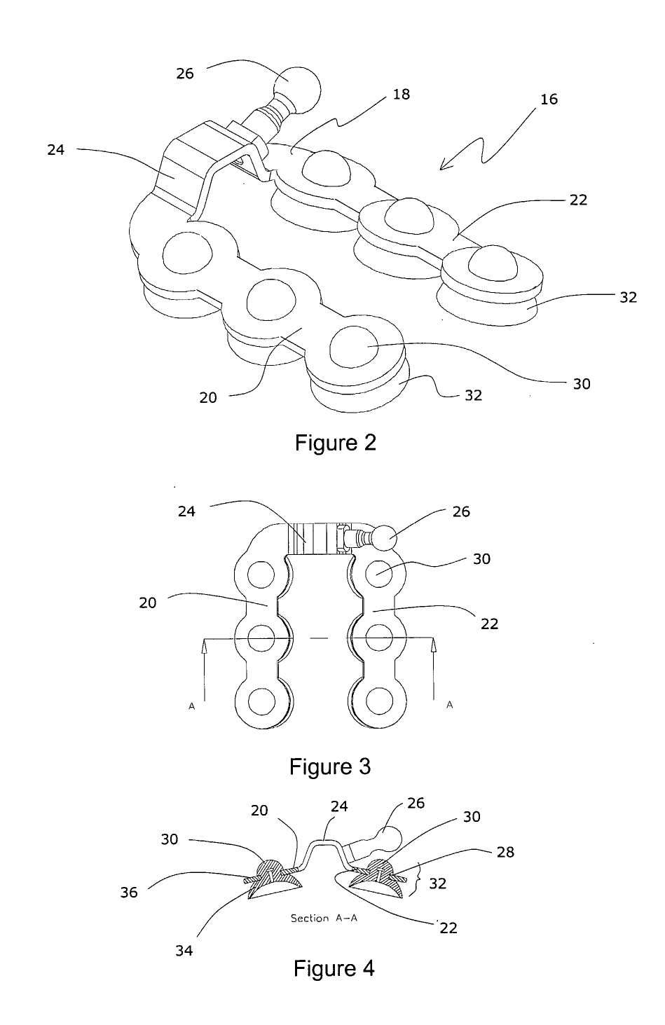

A first embodiment of the suction cup stabilizer foot 16 is shown in perspective view in FIG. 2. The suction cup stabilizer foot 16 includes a frame 18 having first and second arms 20, 22 extending in parallel from a bridge 24 joining proximal ends of first and second arms 20, 22. An attachment ball 26 extends from the frame 18, and more particularly from the bridge 24, to allow for gimbaled attachment of the suction cup stabilizer foot 16 to the distal end 14 of a support arm 10 as described in Publication No. US 2003-0158542 A1. The frame 18, including the arms 20, 22, the bridge 24 and the attachment ball 26 are made of a rigid material, for example, stainless steel. As illustrated in FIG. 2, the bridge 24 has an arch shape extending between the first and second arms 20, 22. It is also within the scope of the present invention that the bridge 24 resides within a single plane between the first and second arms 20, 22 in the manner depicted in FIG. 11A of Publication No. US 2003-0158542 A1. The arched bridge allows the device to be used for anastomosis without occluding a target blood vessel (i.e., vein or artery). The arched bridge may not be necessary because suction cups 32 space the bridge above the target blood vessel to prevent occlusion.

Spaced lengthwise along the first and second arms 20, 22 are a number of holes 28 (see FIG. 4) receiving attachment buttons 30 of suction cups 32. Referring to FIG. 4, the suction cups 32 each consist of a cup 34 with the button 30 extending opposite the cup 34 and an annular groove 36 between the button 30 and the back surface of the cup 34. As depicted in FIG. 4, this configuration of the cup allows the button to be snap fit into the hole 28 with the annular groove 36 receiving an edge of the hole 28 to secure the suction cup 32 to the first and second legs 20, 22. The suction cups 32 may be integrally formed of a biocompatible rubber such as silicone rubber. Forming the suction cups of a material having a durometer of about 50 Shore A has proven effective.

As illustrated in FIG. 4, the first and second legs 20, 22 are canted relative to each other about a lengthwise axis in a concave manner facilitating attachment to convex surfaces.

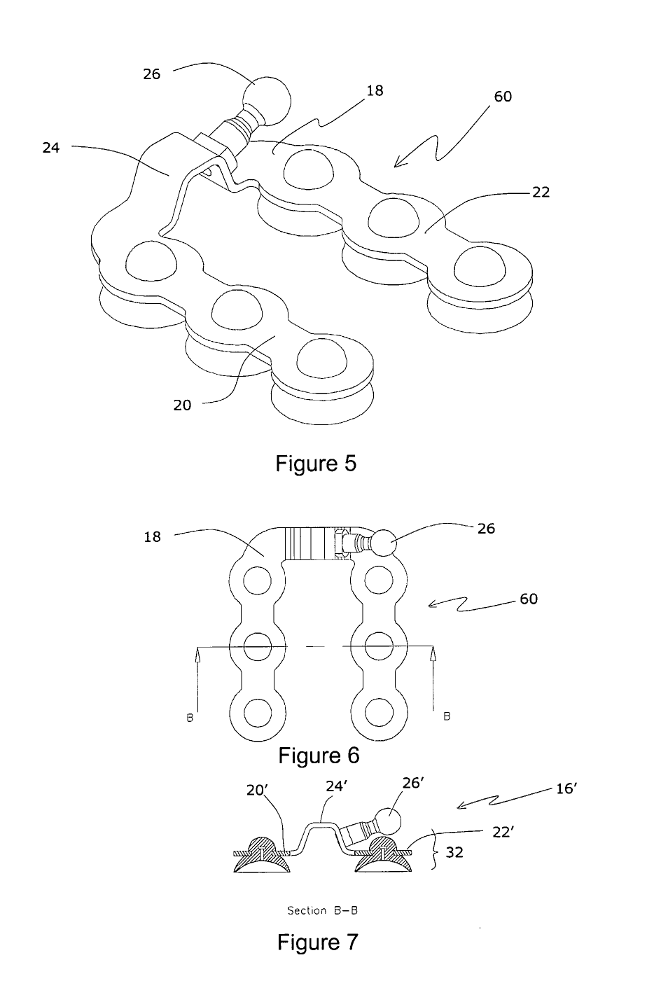

FIG. 5 is a second embodiment of the suction cup stabilizer foot 60 in accordance with the present invention. The second embodiment 60 is substantially identical to the suction cup stabilizer foot 16 illustrated in FIGS. 2-4 and like reference numbers are used to describe like elements. Referring to FIG. 7, the difference between the first embodiment 16 and second embodiment 60 of the suction cup stabilizer foot is in the second embodiment 60 the first and second legs 20, 22 are not canted relative to each other and reside within substantially the same plane. The second embodiment of the suction cup stabilizer foot 60 would therefore be of advantage for stabilizing substantially planar tissue surfaces.

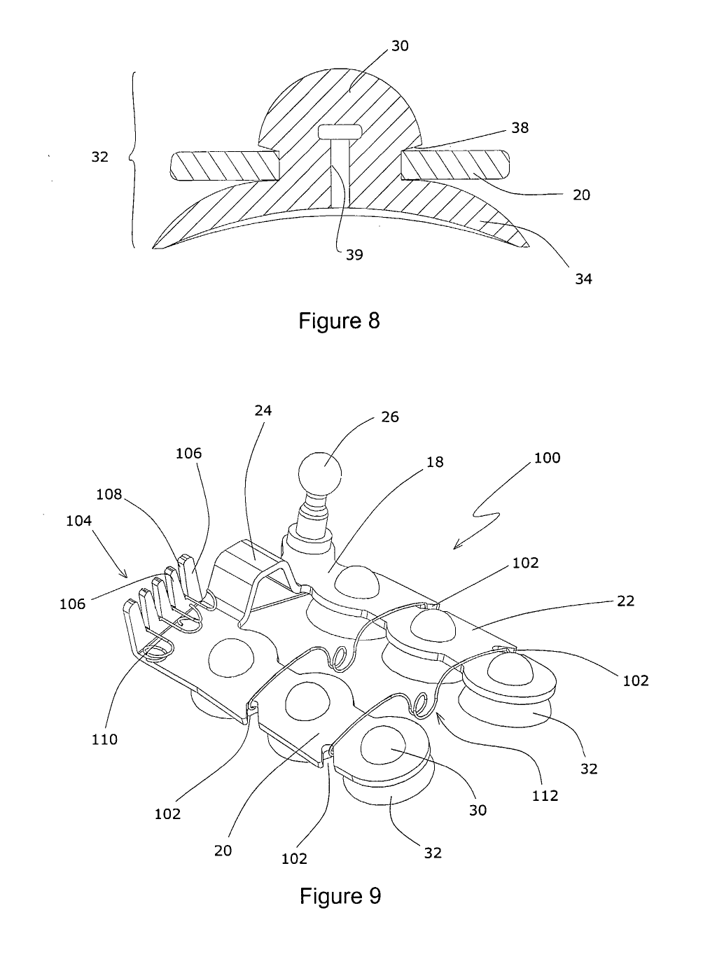

In another alternate embodiment, the suction cups 32 have a gimbaled attachment to the first and second legs of the frame 18. The gimbaled attachment facilitates stabilization of non-uniform epicardial and other tissue surfaces. Such a gimbaled attachment can also obviate the need for canted first and second legs 20, 22 as illustrated with respect to the first embodiment in FIGS. 2-4. The gimbaling could be provided through a relatively simple structure illustrated in FIG. 8 where an underside of the button 30 has an arcuate surface 38 to enable gimbaled movement of the suction cup 32 relative to the frame 18, such as a ball and socket connection. As appreciated by those of skill in the art, any number of substitute attachment structures between the suction cup 32 and the frame 18 could be provided to enable gimbaled movement of the suction cup 32 relative to the frame 18.

Also shown in FIG. 8 is a mushroom-shaped cavity 39 in the wall of the cup 34. This cavity will receive blood or other fluid in use while inhibiting removal of fluid from the cavity. In this manner, the cavity 39 renders the suction cup incapable of adequate sanitization. This feature will encourage disposal of the suction cups and minimize any practice of reusing the suction cups which could present a disease vector and a health hazard. The cavity 39 may be used in any embodiment of the suction cup stabilizer disclosed herein or within the scope of the claims.

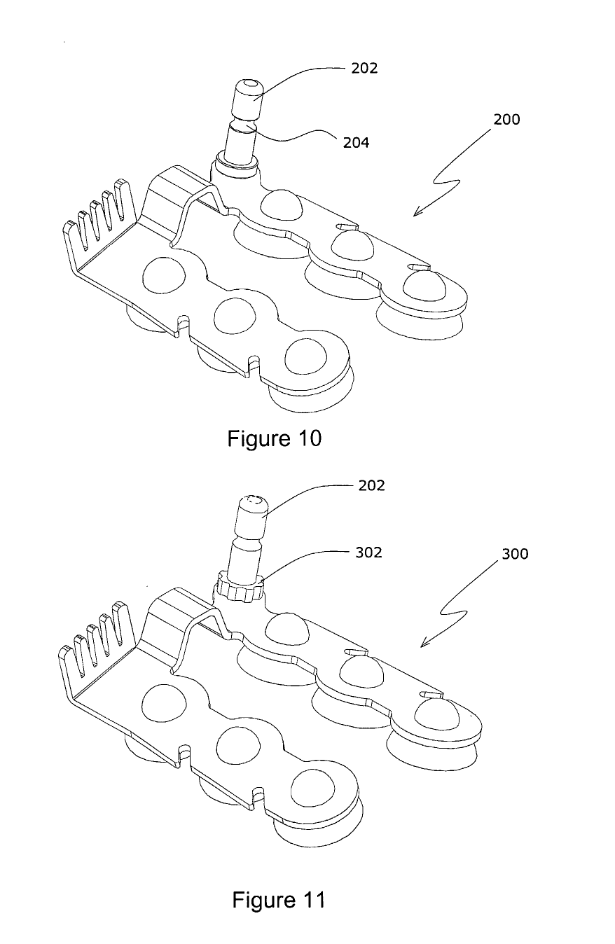

A third embodiment of a suction cup stabilizer 100 in accordance with the present invention is shown in perspective view in FIG. 9. The third embodiment 100 is similar in structure to the suction cup stabilizer foot 16 illustrated in FIGS. 2-4 and like reference numbers are used to describe like elements. The third embodiment differs by providing a number of sutures stays 102 spaced lengthwise of the arms 20, 22. The third embodiment 100 also includes a pinch cleat 104 at a proximal end of the arm 20. The pinch cleat 104 consists of a number of tines 106 having V-shaped slots 108 between adjacent tines 106. The V-shaped slots 108 are configured to pinch a suture 110 therein to retain it in place as illustrated in FIG. 10.

In use, the third embodiment of the suction cup stabilizer foot 100 is oriented with the arms 20, 22 extending lengthwise of a portion of a target artery. One or more vascular loops 110 are formed into loops 112 around axially spaced lengthwise portions of the target artery. The frame 18 is pressed onto the heart to create a partial vacuum between the epicardial tissue and the suction cups, attaching the suction cups to the heart. The vascular loop 110 is then tensioned to occlude the lengthwise portions of the target artery. Ends of the suture are secured within the pinch cleat 104 to maintain the tension in the vascular loop. The act of looping the vascular loop around the target artery not only occludes the target artery, it also serves to elevate the target artery relatively to the epicardium to improve access and to further secure the suction cup stabilizer foot to the surface of the heart.

FIG. 10 is a fourth embodiment of the suction cup stabilizer foot 200 that is substantially identical to the third embodiment 100 of FIG. 10, only the attachment ball 26 is replaced by an attachment post 202. The attachment post 202 has an annular slot 204 which can receive a pin at a distal end of a support arm assembly such as the snout 332 illustrated in FIG. 4C of U.S. Patent Publication No. US 2003-0158542 A1. The attachment post 202 allows the stabilizer foot 200 pivot about the axis of the post 202 but does not permit fully gimbaled movement of the suction cup stabilizer foot 200. The attachment post 202 may be substituted for the attachment ball in any embodiment discussed herein or within the scope of the claims.

FIG. 11 illustrates a fifth embodiment 300 which is substantially identical to the fourth embodiment 200 of FIG. 11. The only difference is that a cog 302 is disposed about the base of the post 202. The cog 302 may be engaged by a structure similar to the snout described above with respect to FIG. 11 to more rigidly secure the fifth embodiment of the suction cup stabilizer 300 against pivoting about the axis of the attachment post 202. Again, this form of the attachment post could be used with any embodiment of the invention.

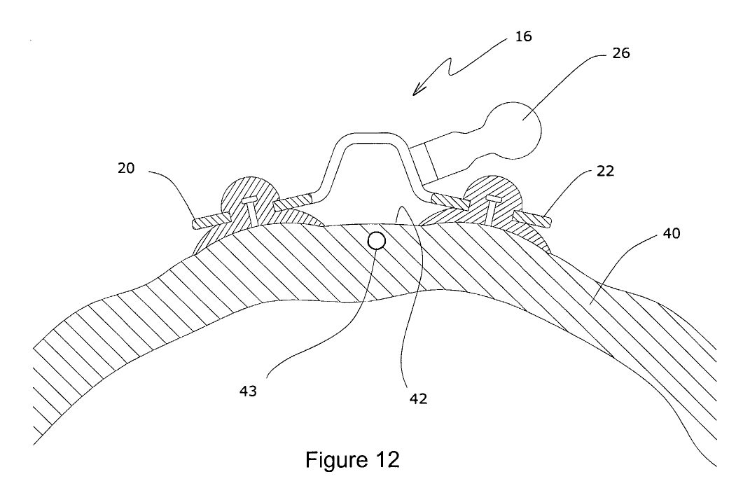

FIG. 12 illustrates the suction cup stabilizer applied to the epicardium 40 of a heart bridging and parallel to a target coronary artery 43. More particularly, the first embodiment 16 with the first and second feet 20, 22 defining an essentially concave relation therebetween is shown adhered to a convex target portion 42 of the epicardium bridging and parallel to a target coronary artery 43. With the suction cup stabilizer foot 16 adhered as illustrated in FIG. 8 and the attachment ball 26 attached to a distal end 14 of a support arm 10 which is in turn attached to a sternal retractor 12 deployed in open heart surgery, the target portion 42 of the epicardium is thereby stabilized notwithstanding continued beating of the heart.

The frame, including the arms, may be made of a malleable material to enable a surgeon to bend and conform the frame to approximate the profile of a target portion of the heart. Such a frame material may be included in all embodiments disclosed herein and within the scope of the claims. One sample material is 304 stainless steel having a thickness of 0.034 inch.

In use, the suction cups are adhered to a tissue surface by placing the mouth of each cup 34 into contact with the tissue surface and exerting a normal force on the frame that causes the cups 34 to distort as illustrated in FIG. 9 and displace air within the cups 34, thereby creating a partial vacuum with the tissue surface. It may be necessary or desired to first wet the perimeter of the cup 34 mouth with a suitable liquid such as saline, glycerol or water to ensure a seal enabling the partial vacuum to secure the tissue.

The first, second, third, fourth and fifth embodiments 16, 60, 100, 200, 300 as described herein have parallel arms 20, 22 (and 20′, 22′) which, as illustrated in FIG. 12, partially surround a target portion 42 of an epicardium. The suction cup stabilizer foot 16 may be suitable for use in stabilizing a target portion of an epicardium if it consists of only a single arm having more than one suction cup attached to and partially surrounding a target portion of an epicardium surface. Other configurations of the suction cup epicardial stabilizer are also considered to be within the scope of the invention, including an annular frame with suction cups deployed therein, an arcuate frame, an L-shaped frame, the generally U-shaped frame of the first and second embodiments 16, 60 (which could also include a suction cup on the bridge 24) as well as any other structure deploying the suction cups performing a stabilizing function on a target portion of the epicardium or other bodily tissue upon which the suction cup stabilizer foot is deployed.

### EXAMPLE

Performance of a suction cup stabilizer substantially as illustrated in FIGS. 2-4 was compared to a Genesee Biomedical ANASTOSURE™ stabilizer, model Number MSS-SRN and a suction cup stabilizer with the suction cups compromised to prevent formation of the partial vacuum. The suction cup stabilizers included six suction cups, each 0.5 inch in diameter and made of a silicon elastomer having a hardness of about Shore 50 A.

Tests were conducted on fresh pig hearts (weighing approximately 380 gm). Perfused pig hearts had a perfusion catheter placed in the left coronary ostia and retained in place by an external suture. The left coronary tree was perfused with room temperature tap water at a pressure of about 75-80 mm Hg, which inflated the left ventricle of the heart. The surface of the heart was moistened.

In those tests using the suction cup stabilizer, the suction cup stabilizer was firmly pressed down onto the left ventricular epicardium causing it to attach and the downward pressure was released.

In those tests using the compromised suction cup stabilizer the procedure was the same, only the suction cups did not attach.

In those tests using the ANASTOSURE™ stabilizer, the method was similar only no weight or the indicated weights were applied vertically to the stabilizer.

Table 1 sets forth a vertical adhesion force produced by the suction cup stabilizer on a perfused pig heart. Table 2 sets forth a vertical adhesion force produced by the suction cup stabilizer on a flaccid pig heart.

Table 3 sets forth a shear force produced by the suction cub stabilizer on a perfused pig heart. The shear force was applied substantially parallel to the legs of the stabilizer in all tests. Table 4 sets forth the shear force produced by a compromised suction cup stabilizer on a perfused pig heart. Table 5 shows a shear force produced by the ANASTOSURE™ stabilizer under a zero and a four ounce applied vertical load.

Tables 3-5 demonstrate significantly improved shear force of the suction cup stabilizer when employed on a perfused pig heart over the compromised suction cup stabilizer and the ANASTOSURE™ stabilizer even under an applied load of 4 ounces.

Table 6 sets forth a shear force of the suction cup stabilizer on a flaccid pig heart under loads of zero and four ounces. Table 7 sets forth a shear force of the compromised suction cup stabilizer on the flaccid pig heart under no load. Table 8 sets forth a shear force of an ANASTOSURE™ stabilizer under vertical loads of zero, four and eight ounces.

While under an applied vertical load of eight ounces, the ANASTOSURE™ stabilizer produced the highest shear force on a flaccid heart, the suction cup stabilizer produced superior shear forces under zero or four ounce loads. Data for shear force of the suction cup stabilizer under a vertical load of eight ounces was not collected.

The suction cup stabilizer of the present invention provides an inexpensive means for stabilizing an epicardial surface without requiring connection to a vacuum source as contemplated in prior art vacuum stabilizers. The suction cup stabilizer provides a suction grip providing a higher degree of stabilization than is possible from friction based devices under minimal vertical loads, such as those devices described and illustrated in Publication No. US 2003-0158542 A1, the Guidant Acrobat Mechanical Off Pump System and comparable systems of U.S. Surgical, Genzyme, Genesee Biomedical and Estech. The suction cup stabilizer can be inexpensively manufactured, thus facilitating its use and the many inherent advantages.

TABLE 1

| Applied Vertical Load oz | Vertical Pull (lbs) |
|---|---|
| 0 | 0.70 |
| 0 | 0.72 |
| 0 | 0.84 |
| 0 | 0.78 |
| 0 | 0.94 |
| 0 | 0.84 |
| 0 | 1.28 |
| 0 | 1.00 |
| 0 | 0.84 |
| 0 | 1.02 |
| | Mean 0.896, SD 0.17 |

TABLE 2

| Applied Vertical Load oz | Vertical Pull (lbs) |
|---|---|
| 0 | 0.22 |
| 0 | 0.44 |
| 0 | 0.18 |
| 0 | 0.36 |
| 0 | 0.38 |
| 0 | 0.16 |
| 0 | 0.40 |
| 0 | 0.38 |
| 0 | 0.38 |
| 0 | 0.28 |
| | Mean 0.32, SD 0.01 |

TABLE 3

| Applied Vertical Load oz | Shear Pull (lbs) |
|---|---|
| 0 | 0.30 |
| 0 | 0.18 |
| 0 | 0.26 |
| 0 | 0.28 |
| 0 | 0.42 |
| 0 | 0.34 |
| 0 | 0.34 |
| 0 | 0.38 |
| | Mean 0.31, SD 0.08 |

TABLE 4

| Applied Vertical Load oz | Shear Pull (lbs) |
|---|---|
| 0 | 0.00 |
| 0 | 0.00 |
| 0 | 0.02 |
| 0 | 0.00 |
| 0 | 0.02 |
| 0 | 0.00 |
| | Mean 0.007, SD 0.01 |

TABLE 5

| Applied Vertical Load oz | Shear Pull (lbs) |
|---|---|
| 0 | 0 |
| 0 | 0 |
| 0 | Mean 0 |
| 4 | 0.14 |
| 4 | 0.20 |
| 4 | 0.20 |
| 4 | 0.26 |
| 4 | 0.20 |
| | Mean 0.20, SD 0.04 |

TABLE 6

| Applied Vertical Load oz | Shear Pull (lbs) |
|---|---|
| 0 | 0.22 |
| 0 | 0.26 |
| 0 | 0.22 |
| 0 | 0.30 |
| 0 | 0.30 |
| 0 | 0.36 |
| 0 | 0.22 |
| 0 | Mean 0.29, SD 0.05 |
| 4 | 0.36 |
| 4 | 0.32 |
| 4 | 0.36 |
| 4 | 0.42 |
| 4 | Mean 0.37, SD 0.04 |

TABLE 7

| Applied Vertical Load oz | Shear Pull (lbs) |
|---|---|
| 0 | 0.02 |
| 0 | 0 |
| 0 | 0.02 |
| 0 | 0 |
| 0 | 0.06 |
| 0 | 0 |
| 0 | 0.04 |
| | Mean 0.02, SD 0.02 |

TABLE 8

| Applied Vertical Load oz | Shear Pull (lbs) |
|---|---|
| 0 | 0 |
| 0 | 0 |
| 0 | Mean 0 |
| 4 | 0.30 |
| 4 | 0.32 |
| 4 | 0.34 |
| 4 | 0.28 |
| 4 | 0.34 |
| 4 | Mean 0.32, SD 0.03 |
| 8 | 0.58 |
| 8 | 0.56 |
| 8 | 0.54 |
| 8 | 0.54 |
| 8 | 0.58 |
| 8 | Mean 0.56, SD 0.02 |

1. An apparatus for stabilizing a target portion of a beating heart comprising:
a frame configured to reside adjacent the target portion of the beating heart; and
at least two suction cups operatively associated with the frame to adhere the frame to a surface of the beating heart adjacent the target portion.

2. The apparatus of claim 1 wherein the frame partially surrounds the target portion.

3. The apparatus of claim 1 further comprising a coupling provided on the frame for gimbaled attachment of the frame to a support arm.

4. The apparatus of claim 3 wherein the coupling is a ball connector providing gimbaled attachment.

5. The apparatus of claim 1 further comprising a coupling on the frame, the coupling being a post connector providing pivoting attachment in a plane normal to the post connector.

6. The apparatus of claim 1 wherein the frame comprises two arms extending substantially in parallel joined at proximal ends to a bridge extending therebetween.

7. The apparatus of claim 6 wherein the bridge defines an arch extending above the first and second arms.

8. The apparatus of claim 6 wherein the two arms are canted relative to each other along a lengthwise axis to define a substantially concave configuration therebetween.

9. The apparatus of claim 6 wherein the two arms are coplanar.

10. The apparatus of claim 1 further comprising a gimbaled attachment between the frame and each suction cup.

11. The apparatus of claim 6 further comprising at least one suction cup on each arm.

12. The apparatus of claim 1 wherein each suction cup is made of a biocompatible material having a durometer of about 50 Shore A.

13. The apparatus of claim 11 further comprising a plurality of suture stays spaced along the arms.

14. The apparatus of claim 13 further comprising a pinch cleat operatively associated with the frame configured to grasp a suture engaged by the suture stays.

15. The apparatus of claim 1 further comprising a cavity operatively associated with an inner surface of each suction cup, the cavity being configured to allow flow of fluid into the cavity while inhibiting removal of fluid from the cavity.

16. The apparatus of claim 1 wherein the frame comprises a malleable material enabling a surgeon to conform the frame to a surface of the beating heart adjacent the target portion.

17. A method for stabilizing a target portion of a beating heart comprising:
providing a frame having at least two suction cups attached to the frame;
pressing the suction cups onto tissue adjacent the target portion of the beating heart to form a partial vacuum attachment therebetween; and
fixing the position of the frame to prevent motion of the frame relative to the beating heart.

18. The method of claim 17 further comprising:
providing suture stays spaced along the frame;
pressing the suction cups to partially surround a lengthwise portion of a target artery;
inserting a vascular loop around axially spaced lengthwise portions of a target artery to form a loop around each axially spaced lengthwise portion of the target artery;
tensioning the vascular loop to occlude the lengthwise portions of the target artery; and
attaching the vascular loop to the suture stays.

19. The method of claim 18 wherein the frame comprises two elongate arms extending substantially in parallel joined at proximal ends to a bridge extending therebetween, at least two of the plurality of suture stays and at least two suction cups being spaced along the length of each lengthwise arm.
